(19) United States
(12) Patent Application Publication
(10) Pub. No.: US 2021/0049734 A1
(43) Pub. Date: Feb. 18, 2021

(54) IMAGE PROCESSING METHOD AND APPARATUS

(71) Applicant: CANON MEDICAL SYSTEMS CORPORATION, Otawara-shi (JP)

(72) Inventors: Magnus WAHRENBERG, Edinburgh (GB); Scott Alan SMITH, Edinburgh (GB); Takahiko NISHIOKA, Otawara (JP); Fumimasa SHIGE, Otawara (JP)

(73) Assignee: CANON MEDICAL SYSTEMS CORPORATION, Otawara-shi (JP)

(21) Appl. No.: 17/064,664

(22) Filed: Oct. 7, 2020

Related U.S. Application Data
(63) Continuation-in-part of application No. 16/538,072, filed on Aug. 12, 2019.

(51) Int. Cl. G06T 3/40; G06T 5/20

(52) U.S. Cl. CPC G06T 3/4007; G16H 30/40; G06T 5/20

(57) ABSTRACT

An image processing apparatus comprises processing circuitry configured to: acquire first image data that is representative of a subject at a first time and second image data that is representative of the subject at a second, different time; process the first image data and second image data to obtain a plurality of transformed first data sets and a plurality of transformed second data sets; transform the transformed first data sets and transformed second data sets to obtain respective first distance transforms and second distance transforms; select a combination of at least one of the first distance transforms and at least one of the second distance transforms; generate at least one morphed distance transform based on the combination; and process the at least one morphed distance transform to obtain upsampled image data that is representative of the subject at a third time.

### CROSS-REFERENCE TO RELATED APPLICATIONS

This application is a continuation-in-part of and claims the benefit of priority from U.S. application Ser. No. 16/538,072, filed on Aug. 12, 2019, the entire contents of which are incorporated herein by reference.

### FIELD

Embodiments described herein relate generally to a method and apparatus for image processing, for example for temporal upsampling of frames.

### BACKGROUND

It is known to perform so-called four-dimensional (4D) medical imaging, for example 4D ultrasound imaging. In 4D imaging systems, a series of three-dimensional (3D) images obtained at different times may be dynamically rendered to produce a moving 3D image, for example a 3D movie.

Each three-dimensional (3D) image may be obtained by using software to combine data that has been taken at different positions or angles to obtain volumetric data, and to render an image from the volumetric data using methods such as simple surface shading or direct volume rendering.

In some circumstances, the number of frames captured in 4D imaging is not enough for an animated playback of the frames to seem smooth to a viewer. The frames may be captured at a rate below that which the human eye perceives as being representative of smooth motion. For example, the frames may be captured at below 10 frames per second, or between 10 and 15 frames per second.

A viewer may find it difficult to view a moving image that does not appear to the viewer to be moving smoothly. For example, a viewer may be distracted by discontinuous motion. A viewer may find it difficult to identify anatomical features or abnormalities in a sequence of images that do not appear to the viewer to be moving smoothly.

There are several use cases in which 4D acquisitions capture multiple 3D data volumes, where each 3D volume presents a point in time or a phase of anatomy.

A first example of a use case is 4D ultrasound. In 4D ultrasound, successive frames of volumetric data are acquired by an ultrasound scanner and rendered for viewing. In an example, ultrasound imaging of a mitral valve is performed at 11 frames per second (fps). 11 fps may be considered to be just under a frame rate that is required for the human eye to perceive the motion between frames as being smooth. If the 4D imaging is displayed at 11 fps, a viewer may perceive the motion of the mitral valve to be jerky or discontinuous. The discontinuous motion may detract from the user's ability to analyses the images displayed. The discontinuous motion may make it difficult to interpret the images.

A second example of a use case is multi-phase CT imaging of a heart. In multi-phase CT imaging, image data is captured that is representative of heart motion over one or more cycles of heart motion, typically multiple cycles of heart motion. The image data is gated into frames that are representative of individual phases of the heart motion. For example, image data may be obtained that is representative of 9 different cardiac phases. The data for the different heart phases may be used to render a sequence of images that is representative of heart motion at different times in a cycle of heart motion.

We consider an example in which images of 9 cardiac phases are played one after the other. A viewer may perceive the apparent motion of the heart as not being smooth. The user's ability to analyses the images may be affected by the lack of smoothness in the motion.

Multi-phase CT or other multi-phase imaging may also be performed in respect of other types of motion.

It is known to interpolate motion between successive image frames by using inter-frame or inter-phase registration. To interpolate motion using registration, image data for a first frame is registered to image data for a second frame, for example an adjacent frame. Any suitable registration method may be used, for example any suitable method of non-rigid registration. The registration maps points on the first frame to corresponding points on the second frame.

Once a registration has been obtained, the registration may be used to obtain a third frame that is representative of a time between the first frame and the second frame. The third frame is obtained by interpolating image values based on the registration.

The data that is interpolated between frames may be 2D or 3D. For example, a 3D motion field produced by a registration may be interpolated.

It has been found that interpolating between frames using registration may result in smooth playback. However, successfully interpolating using registration requires the registration to be accurate. If there is difficulty in obtaining a registration, it may not be possible to generate an interpolated frame. In some circumstances, registration may fail entirely. Requiring that every single registration is performed accurately may be a very demanding requirement.

Interpolating using registration may require a lot of processing and resources. The level of processing and resources that are used in registration may make it difficult to perform a registration-based interpolation in real time. Registration-based interpolation may not be appropriate for some real time applications, for example real time ultrasound imaging. Registration may not deal well with changes in topology.

Different modalities of imaging may require the use of different registration methods. Good registration methods may not be available for some modalities.

For observation of cardiac motion, there may be a need to see among the myocardium, coronary vessels and valves. However, in some phases, the coronary vessels or valve may disappear from view because of the motion of the heart or because of the structure's thickness. In some phases, the coronary vessels may disappear from view due to insufficient concentration of contrast. A valve may disappear from view due to its shape when open.

In some circumstances, registration-based methods may not work well when certain image features (for example, valves) may disappear or reappear from view between successive images.

Another method of obtaining intermediate frames that may be considered is a volume blending technique which blends adjacent frames. For example, one may consider blending a first frame and a second frame to obtain a third frame between the first frame and second frame. Each pixel of the third frame may be allocated a value that is intermediate between its value in the first frame and its value in the second frame.

However, it has been found that simple volume blending techniques do not generally give an appearance of motion. Instead, they may provide a sensation of fading between the frames.

### BRIEF DESCRIPTION OF THE DRAWINGS

Embodiments are now described, by way of non-limiting example, and are illustrated in the following figures, in which:

FIG. 1 is a schematic diagram of an apparatus according to an embodiment;

FIG. 2 is a flow chart illustrating in overview a method of an embodiment;

FIG. 3 is a flow chart showing some of the images generated and used in the embodiment of FIG. 2;

FIG. 4 illustrates an example of a signed distance field;

FIG. 5 illustrates an example of a plurality of iso-level images and a combined image;

FIG. 6 is a flow chart illustrating in overview a method of providing an animation in accordance with an embodiment;

FIG. 7 is a flow chart illustrating in overview a further method of providing an animation in accordance with an embodiment;

FIG. 8 is a schematic illustration of a use of segmentation in an embodiment;

FIG. 9 is a schematic illustration of a further use of segmentation in an embodiment;

FIG. 10 is a schematic illustration of acquisition of successive frames, each acquired at a respective time point;

FIG. 11 is a schematic illustration of acquisition of successive frames, each acquired over a respective extended time period;

FIG. 12 is a schematic illustration of acquisition of two ultrasound frames;

FIG. 13 is a flowchart illustrating an overview a method according to an embodiment;

FIG. 14 is a flow chart illustrating in overview a method of providing an animation in accordance with an embodiment; and

FIG. 15 is a diagram illustrating a simplified data acquisition scenario.

### DETAILED DESCRIPTION

Certain embodiments provide an image processing apparatus comprising processing circuitry configured to: acquire first image data that is representative of a subject at a first time and second image data that is representative of the subject at a second, different time; process the first image data based on a parameter of the image data to obtain a plurality of transformed first data sets, each of the transformed first data sets corresponding to a respective value for the parameter; process the second image data based on the parameter of the image data to obtain a plurality of transformed second data sets, each of the transformed second data sets corresponding to a respective value for the parameter; transform each of the transformed first data sets to obtain a respective first distance transform; transform each of the transformed second data sets to obtain a respective second distance transform; select a combination of at least one of the first distance transform and at least one of the second distance transforms based on the parameter; generate at least one morphed distance transform based on the combination; and process the at least one morphed distance transform to obtain upsampled image data that is representative of the subject at a third time, wherein the third time is between the first time and the second time.

Certain embodiments provide an image processing method comprising: acquiring first image data that is representative of a subject at a first time and second image data that is representative of the subject at a second, different time; processing the first image data based on a parameter of the image data to obtain a plurality of transformed first data sets, each of the transformed first data sets corresponding to a respective value for the parameter; processing the second image data based on the parameter of the image data to obtain a plurality of transformed second data sets, each of the transformed second data sets corresponding to a respective value for the parameter; transforming each of the transformed first data sets to obtain a respective first distance transform; transforming each of the transformed second data sets to obtain a respective second distance transform; selecting a combination of at least one of the first distance transforms and at least one of the second distance transforms based on the parameter; generating at least one morphed distance transform based on the combination; and processing the at least one morphed distance transform to obtain upsampled image data that is representative of the subject at a third time, wherein the third time is between the first time and the second time.

Certain embodiments provide an image processing apparatus comprising processing circuitry configured to: acquire first image data that is representative of a subject at a first time and second image data that is representative of the subject at a second, to different time; process the first image data based on a parameter of the image data to obtain a plurality of transformed first data sets, each of the transformed first data sets corresponding to a respective value for the parameter; process the second image data based on the parameter of the image data to obtain a plurality of transformed second data sets, each of the transformed second data sets corresponding to a respective value for the parameter; identify a defect of the first image data and/or the second image data, wherein the identifying of the defect is based on the first transformed data sets and the second transformed data sets; and generate video data based on the identifying of the defect.

Certain embodiments provide an image processing method comprising: acquiring first image data that is representative of a subject at a first time and second image data that is representative of the subject at a second, different time; processing the first image data based on a parameter of the image data to obtain a plurality of transformed first data sets, each of the transformed first data sets corresponding to a respective value for the parameter; processing the second image data based on the parameter of the image data to obtain a plurality of transformed second data sets, each of the transformed second data sets corresponding to a respective value for the parameter; identifying a defect of the first image data and/or the second image data, wherein the identifying of the defect is based on the first transformed data sets and the second transformed data sets; and generating video data based on the identifying of the defect.

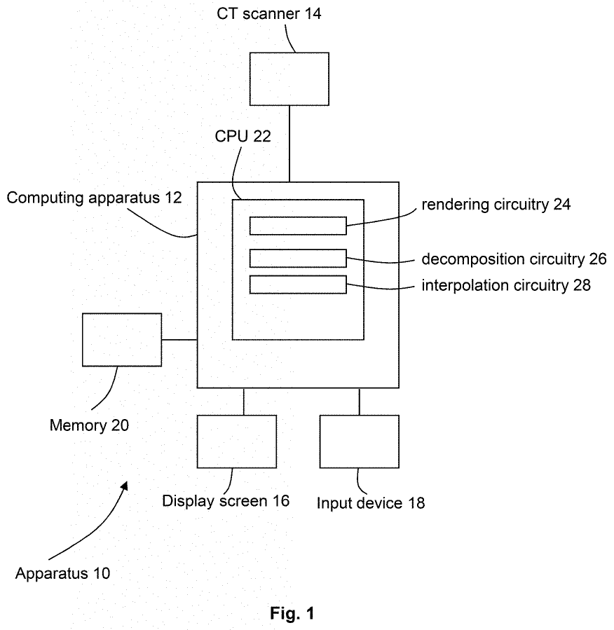

A medical image processing apparatus 10 according to an embodiment is illustrated schematically in FIG. 1.

The apparatus 10 comprises a computing apparatus 12, in this case a personal computer (PC) or workstation, which is connected to a computed tomography (CT) scanner 14, one or more display screens 16 and an input device or devices 18, such as a computer keyboard, mouse or trackball.

The CT scanner 14 is configured to obtain volumetric CT imaging data that is representative of an anatomical region of a patient or other subject. The CT scanner 14 is configured to obtain volumetric CT imaging data over time, while movement is occurring in the anatomical region. For example, the anatomical region may be the to heart and imaging data may be obtained during motion of the heart.

In alternative embodiments, the CT scanner 14 may be replaced or supplemented by a scanner configured to obtain volumetric imaging or two-dimensional imaging data in any appropriate imaging modality, for example a CT scanner, cone-beam CT scanner, MRI (magnetic resonance imaging) scanner or ultrasound scanner.

The imaging data may be obtained using a contrast agent. For example, the imaging data may comprise contrast CT imaging data, or ultrasound data obtained using bubble contrast.

Imaging data sets obtained by the CT scanner 14 may be stored in memory 20 and subsequently provided to computing apparatus 22, or may be provided to computing apparatus 22 directly. In an alternative embodiment, imaging data sets are supplied from a remote data store (not shown) which may form part of a Picture Archiving and Communication System (PACS). The memory 20 or remote data store may comprise any suitable form of memory storage.

Computing apparatus 12 provides a processing resource for automatically or semi-automatically processing imaging data sets, and comprises a central processing unit (CPU) 22.

The computing apparatus 12 includes rendering circuitry 24 configured to render image frames from the volumetric CT imaging data, decomposition circuitry 26 configured to obtain signed distance fields for the image frames, and interpolation circuitry 28 configured to use the signed distance fields to interpolate between the image frames.

In the present embodiment, the circuitries 24, 26, 28 are each implemented in computing apparatus 12 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, to and hardware devices including a graphics card. Such components are not shown in FIG. 1 for clarity.

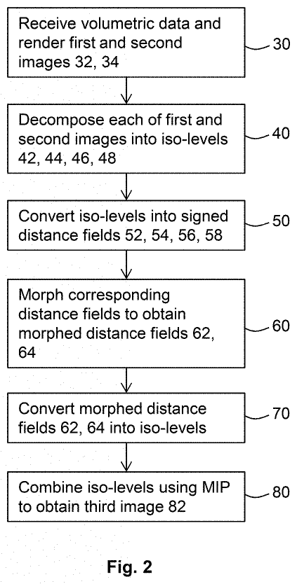

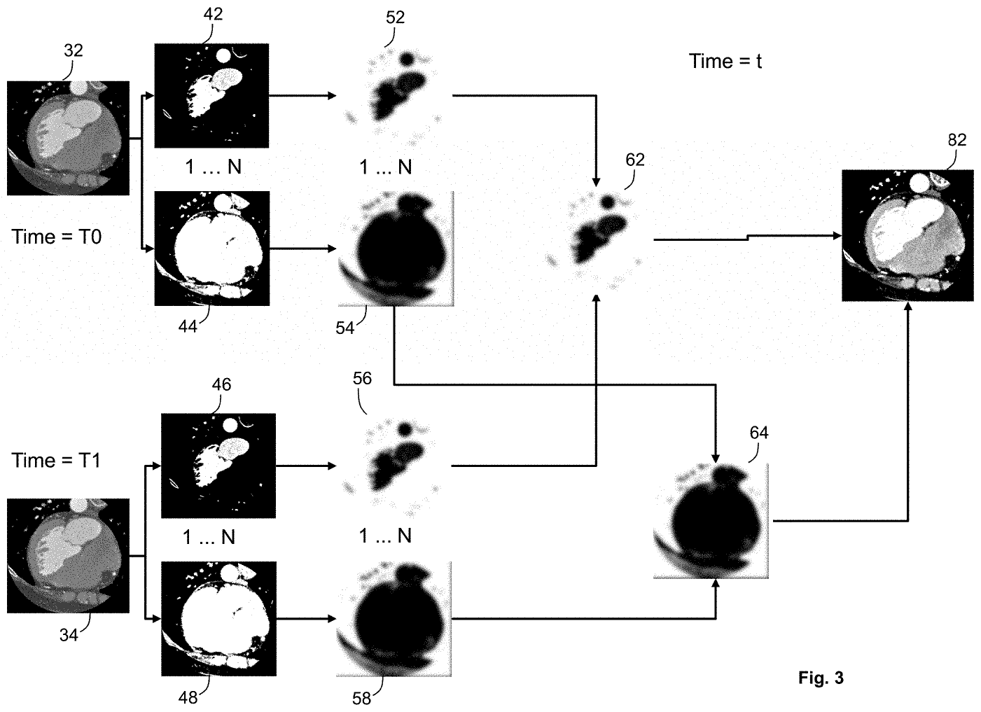

FIG. 2 is a flow chart illustrating in overview a method of an embodiment. FIG. 3 is a further flow chart showing some of the images generated and used in the embodiment of FIG. 2.

At stage 30 of FIG. 1, the rendering circuitry 24 receives volumetric imaging data from the memory 20. The volumetric imaging data is representative of the motion of an anatomical region over time. In further embodiments, the data received by the rendering circuitry 24 may be two-dimensional.

The rendering circuitry 24 renders a first image frame 32 that is representative of the anatomical region at a first time T1, and a second image frame 34 that is representative of the anatomical region at a second time T1. Times T1 and T1 may also be referred to as temporal locations. Movement of the anatomical region has occurred between the first time T1 and the second time T1. The first image frame 32 and second image frame 34 may be adjacent frames of a sequence of frames.

First image frame 32 and second image frame 34 are illustrated in FIG. 3. In the example of FIG. 3, the rendered images 32, 34 are representative of slices of the anatomy, for example axial slices. In other embodiments, the rendered images may be any suitable image type. For example, the rendered images may be images that provide a three-dimensional view of the anatomy. Any suitable rendering method may be used. For example, the rendering method may comprise at least one of Multiplanar Reformat (MPR), Full Volume Intensity Projection volume rendering, Shaded Volume Rendering, Global Illumination Volume Rendering, Curved Plane Reformats (CPR).

Although we describe the process performed by the rendering circuitry 24 in terms of the creation of images, in most embodiments the images 32, 34 are not displayed to the user at this stage. The images 32, 34 are represented by two-dimensional data sets comprising respective pixel values for a plurality of pixel positions. Although we refer below to the processing of images, in practice the processing is performed on the two-dimensional data sets that are representative of the images. Similarly, the iso-levels and signed distance fields described below are illustrated in FIG. 3 only for the purposes of explanation. In most embodiments, the iso-levels and signed distance fields are not displayed to a user. Two-dimensional data sets representative of the iso-levels and signed distance fields are processed by the various circuitries.

It may be seen in FIG. 3 that the rendered images 32, 34 each comprise pixels having a range of different greyscale levels. Different greyscale levels are representative of different CT values, for example CT values in Hounsfield units.

At stage 40, the decomposition circuitry 26 receives a set of N pre-defined iso-level values. In the present embodiment, N=10. In other embodiments, any suitable value for N may be used. The iso-level values are representative of intensity and may comprise, for example, values in Hounsfield units.

In the present embodiment, the set of iso-levels to be used is manually selected by a user. In other embodiments, the set of iso-levels to be used may be determined automatically. The iso-levels may be selected by selecting the most visible parts of the volume. A determination of which parts of the volume are most visible may be obtained using a visibility histogram based on the view presented to the user.

In some embodiments, at least one of the iso-levels may be obtained using a histogram analysis in which a midpoint between significant peaks of the histogram is determined.

In further embodiments, a trained model (for example, a deep neural network, which may comprise a convolutional neural network) may be used to obtain the iso-levels to be used. For example, the trained model may classify the iso-levels that cause the least amount of differences to the final image at key frames. The trained model may have an input volume which comprises at least one key frame. From the at least one key frame, the trained model may regress a fixed vector of thresholds. The loss function may be based on a degree of similarity between a rendered view of the generated volume (based on the proposed decomposition and reconstruction) and a rendered view of a reference source key frame.

The decomposition circuitry 26 decomposes the first rendered image 32 into a first plurality of N iso-level images. The iso-level images are numbered from 1 to N. Each of the N iso-level images is representative of a respective iso-level surface in the first image 32.

In the present embodiment, each of the iso-level images is obtained by thresholding the first rendered image using a respective one of the set of N pre-defined iso-level values. In other embodiments, any suitable method may be used to obtain the iso-level images. In some embodiments, segmentation and/or clipping may be used to constrain each iso-level.

Two examples of iso-level images 42, 44 obtained from the first rendered image 32 are shown in FIG. 3. The iso-level images may also be referred to as volume masks.

The decomposition circuitry 26 decomposes the second rendered image 34 into a second plurality of iso-level images. In the present embodiment the same set of N iso-level values are used for the second plurality of iso-level images as were used for the first plurality of iso-level images. Two examples of iso-level images 46, 48 obtained from the second rendered image 34 are shown in FIG. 3.

At stage 50, the decomposition circuitry 26 converts each of the iso-level images into a respective signed distance field. For each iso-level image, the signed distance field is representative of the shape of the iso-level. In other embodiments, any distance transform may be used that is representative of the shape of the iso-level.

A signed distance field (in three dimensions) is a continuous function [symbol] ³→ [symbol] having values which are representative of a distance to a boundary of an object. The signed distance field may be written as f(x,y,z). The value of f(x,y,z) at each point within the interior of the object is the negated minimum distance from the point to the boundary of the object. f (x,y,z)<0 represents the interior of the object. f (x,y,z)>0 represents the exterior of the object. The value of f(x,y,z) at each point exterior to the object is the minimum distance from the point to the boundary.

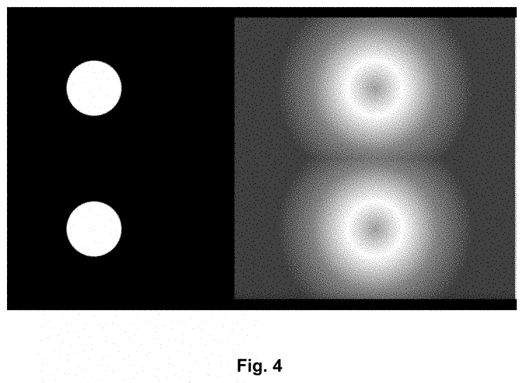

FIG. 4 is a schematic illustration of a signed distance field in two dimensions. The left side of FIG. 4 shows two circles. The right side of FIG. 4 visualizes a distance field for the two circles. The distance field is visualized using a transfer function that maps f (x,y,z)=0 to white and maps other values for f(x,y,z) to different shades of grey. FIG. 4 demonstrates that unconnected shapes (the circles) may be represented as a single distance function.

The output of stage 50 comprises a first plurality of N signed distance fields corresponding to the first plurality of iso-levels obtained from the first rendered image 32. Two of the first plurality of signed distance fields are shown in FIG. 3 as signed distance fields 52, 54.

The output of stage 50 further comprises a second plurality of N signed distance fields corresponding to the second plurality of iso-levels obtained from the second rendered image 34. Two of the second plurality of signed distance fields are shown in FIG. 3 as signed distance fields 56, 58.

The first plurality of signed distance fields and second plurality of signed distance fields are stored temporarily in data store 20 or in any appropriate memory.

In stages 60 to 80 of FIG. 2, the interpolation circuitry 28 uses the signed distance fields to obtain an interpolated volume for a time t in between T0 and T1. Time t may also be referred to as a temporal location between T0 and T1.

At stage 60, the interpolation circuitry 28 morphs corresponding signed distance fields to obtain morphed distance fields. For each corresponding iso-level for the frames at T0 and T1, a new signed distance field is obtained for time t by interpolating the signed distance fields for T0 and T1. A shape blending operation is performed using the temporal location t to calculate the weight of each frame.

Signed distance field morphing is a technique which may be used to volumetrically smooth morph between two shapes without explicitly modelling the motion of points between shapes. In the present embodiment, volumetric morphing is performed on two-dimensional images. It is described as being volumetric because it is applied to two dimensions of position and one dimension of signed distance.

Signed distance morphing may be used to move in between shapes that do not have a simple analytic mapping. For example, morphing may be performed between a rounded cube and a sphere even through no simple analytic mapping between the rounded cube and sphere is available. It has been found that signed distance morphing may deal well with almost all geometry, including disjoint objects.

The morphing of the signed distance fields may be performed using any suitable interpolation method, for example linear, quadratic or cubic interpolation or any similar smooth interpolation method.

In the present embodiment, the interpolation circuitry 28 combines pairs of signed distance fields that correspond to the same iso-level. For example, signed distance field 52 has been obtained from iso-level image 42. Iso-level image 42 is a first iso-level of first image 32 at time T0. Signed distance field 56 has been obtained from iso-level image 46. Iso-level image 46 is a first iso-level of second image 34 at time T1, for the same iso-level value as iso-level image 42.

Signed distance fields 52, 56 are combined by morphing the signed distance fields 52, 56 based on a time t that is between T0 and T1. The combination of signed distance fields 52, 56 is a morphed signed distance field 62.

Similarly, signed distance fields 54 and 58 have been obtained from iso-level images 44 and 48 respectively, for a second iso-level value. Signed distance fields 54, 58 are combined by morphing the signed distance fields 54, 58 based on the time t. The combination of signed distance fields 54, 58 is a morphed signed distance field 64.

Although only two morphed signed distance fields 62, 64 are illustrated in FIG. 3, in practice each corresponding pair of signed distance fields is morphed together, wherein each corresponding pair of signed distance fields comprises signed distance fields that have been obtained from iso-level images having the same iso-level value.

In further embodiments, at least some of the pairs of signed distance fields that are morphed together may have been obtained from iso-level images having different iso-level values. Morphing together images having different iso-level values may be used to account for inconsistencies in the intensity of captured tissue. For example, the same type of tissue may appear to have different intensity values in different images. One reason for an inconsistency in intensity values may be that contrast is fading out. In some circumstances, morphing together images that are representative of the same type of tissue may comprise combining images having different intensity values. In some embodiments, the iso-levels to be combined may be estimated by the model. In some embodiments, the iso-levels to be combined may be selected by a user.

At stage 70, the interpolation circuitry 28 converts each morphed signed distance field back into an iso-level image (which may also be referred to as a volume mask) having an associated iso-level value.

At stage 80, the interpolation circuitry 28 accumulates all of the iso-level images of stage 70 into a single volume 82. In the present embodiment, the iso-level images are accumulated by selecting the maximum intensity between a current morphed iso-level and a previous morphed iso-level. The interpolation circuitry 28 selects the maximum intensity of each morphed iso-level, for each voxel. For every voxel value, we get a set of values representing each iso-level (interpolated shape) after mapping the distance field back into a value space. The interpolation circuitry 28 iterates through the iso-levels to obtain the maximum value. For each iso-level, the interpolation circuitry 28 compares a current voxel value to a previous voxel value, wherein the current value is from the data set for the iso-level currently being considered, and the previous value is the maximum value as determined from the previously-considered iso-levels. The interpolation circuitry 28 selects the maximum of the current value and the previous value.

In other embodiments, any suitable method may be used to combine the iso-level images. For example, the over operator may be used.

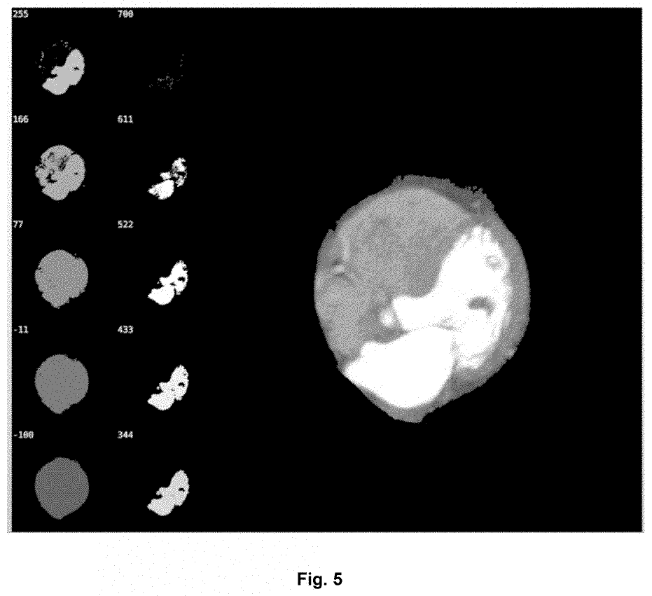

FIG. 5 shows an example of a plurality of iso-level images that are combined into a combined image. The iso-level images may be morphed iso-level images as obtained at stage 70. The iso-level images are obtained for Hounsfield values of −180, −11, 77, 166, 255, 344, 433, 522, 611 and 700 respectively. Each of the iso-level images is reference by its Hounsfield level in FIG. 5.

FIG. 5 also shows a combined image 90 in which all of the individual iso-level images of FIG. 5 are combined into a single image (which in example of FIG. 5 is a slice).

The method of FIG. 2 may provide an easy to use, efficient temporal up-sampling technique that gives a good perception of motion between frames. The method of FIG. 2 may be particularly useful for sequences that are captured in the range of frame rates between 10 fps and 20 fps.

The resources required to perform the morphing process of FIG. 2 may be much less than those required to provide, for example, a registration-based method of interpolation. It has been found that it is difficult for the morphing algorithm to fail. The process of FIG. 2 may provide a robust method of obtaining intermediate frames by interpolation. In general, morphing is a much simpler algorithm than registration. The method of FIG. 2 may be computed efficiently, for example on the GPU.

In some circumstances, the method of FIG. 2 may allow a lower frame rate to be used than would otherwise be the case, which may reduce a resource requirement.

In some circumstances, the method of FIG. 2 may be used to trade off temporal resolution and spatial resolution. For example, it may be the case that the scanner may be configured to capture frames either at high spatial resolution and low frame rate, or at low spatial resolution and high frame rate. The method of FIG. 2 may provide an appearance of increased frame rate due to interpolation, while using images that are acquired at high spatial resolution and low frame rate.

The method of FIG. 2 may accommodate features that appear in view and disappear from view during motion, for example heart valves. The method of FIG. 2 may be applicable to a range of different modalities.

In the embodiment described above with reference to FIG. 2, a single intermediate frame at time t is interpolated between a first frame at time T0 and a second frame at time T1. In other embodiments, multiple intermediate frames may be interpolated at different times between the first frame and second frame. Intermediate frames for different times may be obtained by different weightings of the combination between the signed distance fields of the first frame and the signed distance fields of the second frame.

In the embodiment described above with reference to FIG. 2, the frames are decomposed into iso-level images. In other embodiments, any suitable decomposition may be used. For example, a set of images may be obtained using segmentation and/or clipping. Iso-level images may be used as a starting point for obtaining a segmentation. A segmentation mask may be used alongside the iso-level. The segmentation mask may exclude parts of the volume from being part of the decomposed shapes.

In some embodiments, the decomposition circuitry 26 divides the volume into multiple shapes within each iso-level. Within one target iso-level, the decomposition circuitry 26 may divide the volume into multiple shapes. Each of the multiple shapes may be individually morphed and combined as the interpolation circuitry 28 reconstructs the upsampled volume.

For example, in the case of heart imaging, the vessels overlap in iso-range with the blood pool in the heart. In some embodiments, the vessels are segmented and separated into separate shapes from the main heart, even if the vessels share the same or similar iso-level as the main blood pool.

In embodiments described above, the iso-levels are obtained from a single volume per frame. In other embodiments, iso-levels may be obtained from multiple volumes. For example, the volumes may be obtained using different modalities. The volumes may be obtained from different scans in the same modality, for example with and without contrast. Iso-levels from multiple volumes may be reconstructed into a motion interpolated fusion volume.

In some embodiments, a signed distance field may be included to incorporate an shape that is not part of the anatomy that has been imaged. This shape may be referred to as an external shape. The external shape may be representative of an object or device that is to be introduced to the anatomy. For example, the external shape may be an implant model. The external shape may be any appropriate type of shape, for example a mesh object or mathematical shape.

The signed distance field may be static or animated. The signed distance field may be estimated by a model or user selected.

The external shape may be fused with the volume data and become part of the up-sampled volume. Including the external shape in the up-sampling may make it easier to include the external shape in the volume rendering.

We turn to the question of obtaining an animated view from a sequence of frames. We consider the case in which an initial sequence of frames is obtained at a frame rate that may be insufficient to provide an animation that appears smooth to the user. The method of FIG. 2 is used to interpolate between adjacent pairs of frames.

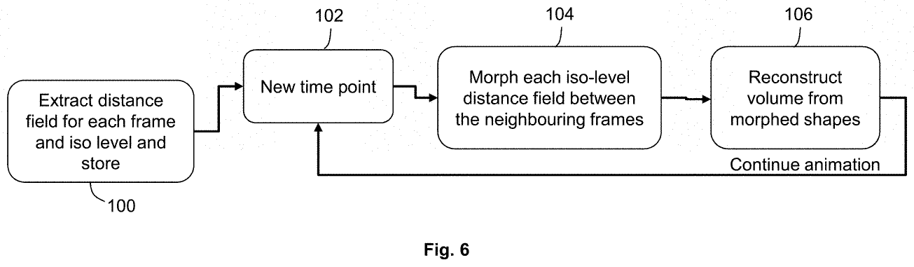

FIG. 6 is a flow chart illustrating in overview a method of providing an animation in accordance with an embodiment.

At stage 100 of FIG. 6, the rendering circuitry 24 receives data that is representative of a sequence of frames. The sequence of frames has been captured at a frame rate that may be insufficient to provide an animation that appears smooth to the user if no interpolation between frames is performed.

The rendering circuitry 24 renders a respective image from each of the sequence of frames. For each image, the decomposition circuitry 26 extracts signed distance fields for each of a plurality of iso-level values. The decomposition circuitry 26 stores the signed distance fields in the data store 20 or in any appropriate memory.

At stage 102, the interpolation circuitry 28 moves to a new time point (for example, a first time point) which is between two adjacent frames of the sequence of frames. At stage 104, the interpolation circuitry 28 morphs each signed distance field for each iso-level of the two adjacent frames to obtain a morphed signed distance field. At stage 106, the interpolation circuitry reconstructs an intermediate frame (which may also be referred to as a volume) from the morphed signed distance fields as described above with reference to stages 70 and 80 of FIG. 2.

The flow chart then returns to stage 102 and a new time point is selected. In some embodiments, the interpolation circuitry 28 may perform a morphing process for a single time point between each pair of adjacent frames, to obtain a single intermediate frame between each pair of adjacent frames in the sequence. In some embodiments, the interpolation circuitry 28 may perform morphing processes for a multiple time points between each pair of adjacent frames, to obtain multiple intermediate frames between each pair of adjacent frames in the sequence.

The interpolation circuitry 28 outputs a set of video data comprising the image data for the original frames and for the interpolated frames. The rendering circuitry 24 displays the resulting animation. The animation comprises the original sequence of frames and the intermediate frames that have been generated during the process of FIG. 6. The animation may appear to a user to be smoother than an animation produced from the original sequence of frames.

In the method of FIG. 6, the decomposing of the original volumes into signed distance fields is performed ahead of time and the result cached. Performing the decomposition ahead of time may reduce the processing resources required during animation.

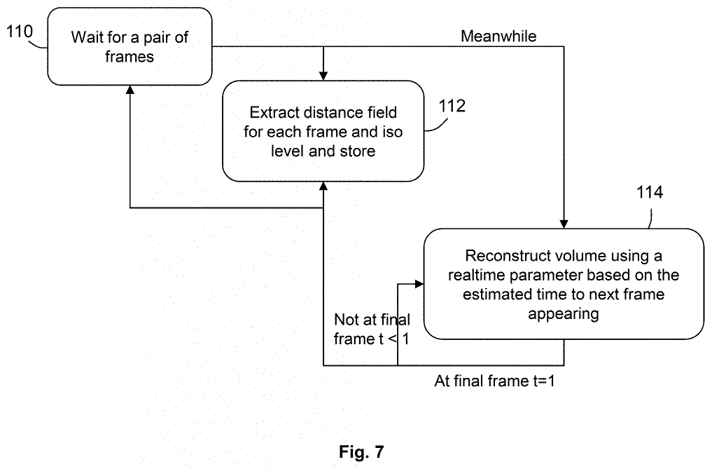

FIG. 7 is a flow chart illustrating in overview a method of providing an animation in accordance with a further embodiment. In the embodiment of FIG. 6, the entire sequence of frames to be animated is already available at the beginning of the animation process. In the method of FIG. 7, the animation is performed on frames that are being received in real time. The algorithm of FIG. 2 is applied in a live mode by performing an animation at one frame behind real-time.

At stage 110, the decomposition circuitry 26 waits for a pair of frames to be received. At stage 112, the decomposition circuitry 26 extracts signed distance fields for each of a plurality of iso-level values for each of the pair of frames. The decomposition circuitry 26 stores the signed distance fields in data store 20 or in any appropriate memory.

In the method of FIG. 7, the decomposition of each pair of images into signed distance fields is performed just in time and the result subset required for a single interpolation at time t is cached.

At stage 114, the interpolation circuitry reconstructs a volume using a real time parameter based on an estimated time to a next frame appearing.

For example, consider a case in which a previous frame was acquired at t=10 s and a new frame came in at t=13 s. If a target frame rate is 30 fps (for example, having hardware that could reconstruct an image in 1/30th of a second), the interpolation circuitry 28 generates 90 frames and displays them at 30 fps, no matter when the next frame is expected. The time estimation may be such as to provide delayed real time playback.

The method of FIG. 7 provides an interpolated frame between the last set of volume frames using the method of FIG. 2 and at a speed based on an expectation of when the next frame will appear.

The method of FIG. 7 may provide an appearance of smooth real time motion which is offset behind the true real time results by an acquisition time step. The method of FIG. 7 may provide a smooth real time display at the price of a slight increase in latency. The increase in latency is the result of rendering being performed at one frame behind real time rather than in real time.

The method of FIG. 7 may be particularly useful in modalities such as ultrasound. In some circumstances, the method of FIG. 7 may involve very heavy processing. The processing may be performed using the GPU.

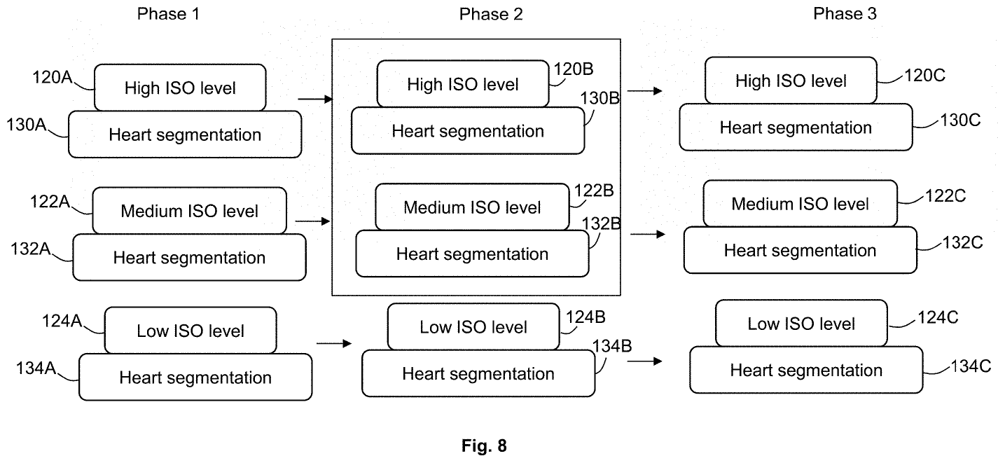

FIG. 8 is a schematic illustration of a use of segmentation in an embodiment. Segmentation may be used to separate the interpolation behavior of individual anatomy. In some circumstances, anatomy may appear and disappear in different phases of motion. For example, in heart motion, it may be expected that a valve will appear and disappear over the motion cycle.

In other circumstances, disappearance of anatomy may be unwanted. For example, the vessels may not be perfectly visible in one cardiac phase. One reason for poor vessel visibility may be lack of contrast. Another may be the presence of imaging artifacts. For example, the coronary artery may typically be more influenced by motion artifact compared to myocardium. This may lead to a lack of the coronary artery in volume data in some phases.

It may preferable if a phase having poor vessel visibility is not used in interpolation. If the anatomy isn't represented in a particular phase, then interpolation using that phase may make the anatomy disappear.

A segmentation in which an anatomy is not correctly represented may be described as a poor segmentation, or as a defect. Any suitable method may be used to determine that a defect is present. In some embodiments, constraints are used to determine whether a defect is present. For example, to determine whether a vessel segmentation is poor, a constraint on vessel volume may be used to determine whether the vessels have an expected volume.

FIG. 8 shows three examples of phases: Phase 1, Phase 2 and Phase 3.

In an embodiment, the decomposition circuitry 26 obtains a high iso-level image 120, medium iso-level image 122 and low iso-level image 124 for each phase. The decomposition circuitry 26 uses the high, medium and low iso-levels as starting points for a first heart segmentation 130 (high iso-level), a second heart segmentation 132 (medium iso-level) and a third heart segmentation 134 (low iso-level). The images and segmentations are denoted by 120A, 122A, 124A, 130A, 132A, 134A for Phase 1; 120B, 122B, 124B, 130B, 132B, 134B for Phase 2; and 120C, 122C, 124C, 130C, 132C, 134C for Phase 3.

The decomposition circuitry 26 assesses whether there is a defect in any of the segmentations. In the example shown, it is found that the heart segmentation is good at all iso-levels in Phase 1 and Phase 3 (130A, 132A, 134A, 130C, 132C, 134C). The heart segmentation is also good for the low iso-level 134B of Phase 2. However, the heart segmentation at the high and medium iso-levels 130B, 132B of Phase 2 is poor. It may be considered that a defect has been identified in Phase 2.

In some embodiments, the interpolation circuitry 28 omits the high iso-level and medium iso-level of Phase 2 from an interpolation process, for example a morphing process as described above with reference to FIG. 2. In some circumstances, an interpolation may be performed directly between Phase 1 and Phase 3 without using Phase 2.

The high iso-level and medium iso-level of Phase 2 may be omitted from the displayed images. For example, the interpolation circuitry 28 may use an interpolation between Phase 1 and Phase 3 in place of the high iso-level and medium iso-level of Phase 2. Alternatively, the interpolation circuitry 28 may use high iso-level and medium iso-level of Phase 1 (or of Phase 3) in place of the high iso-level and medium iso-level of Phase 2.

The interpolation circuitry 28 outputs a set of video data comprising a version of the frames in which any identified defects are omitted (for example, substituted with data from another frame or with interpolated data). The rendering circuitry 24 displays an animation that is a rendering of the output video data.

Omitting parts of images with poor visibility may result in a better overall animation. A better interpolation may be obtained if images with poor visibility of certain anatomy are not used in the interpolation process. The interpolation circuitry 28 may exclude frames in which a segmented anatomy is not well represented. For example, the heart may interpolate across 9 phases but the vessels may interpolate across 6 phases. All of the phases may be combined into one destination volume.

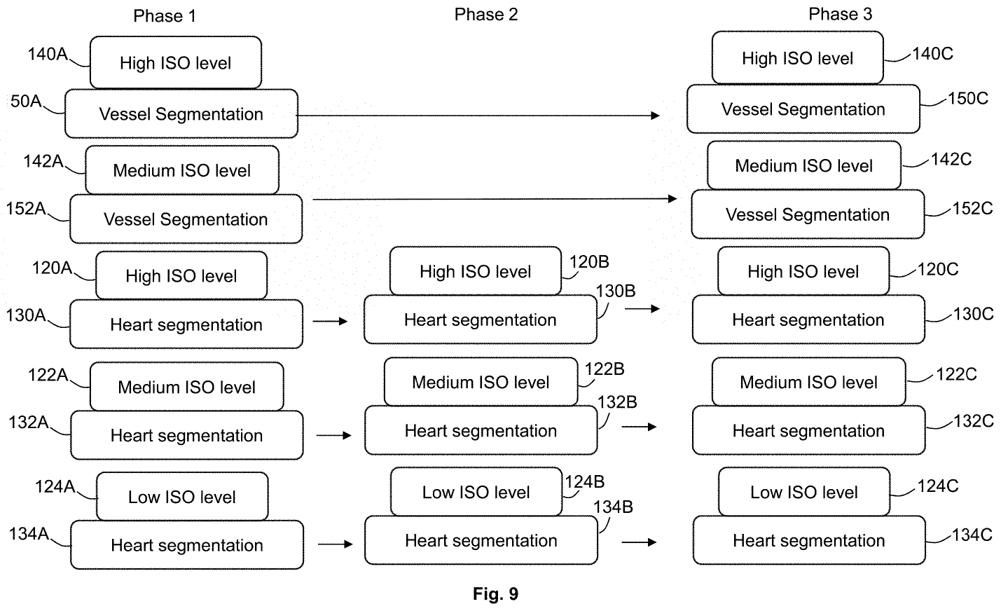

FIG. 9 is a schematic illustration of a further use of segmentation in an embodiment. In the embodiment of FIG. 9, no interpolation is performed.

The decomposition circuitry 26 obtains heart segmentations are obtained for high, medium and low iso-levels for each frame as shown in FIG. 8. Any suitable segmentation method may be used. In the embodiment of FIG. 9, all of the heart segmentations are found to be good and no defects are identified.

The decomposition circuitry 26 also obtains vessel segmentations 150, 152 are also obtained for a high iso-level 140 and medium iso-level 142 respectively, using any appropriate segmentation method.

In the example shown in FIG. 9, the vessel segmentations for Phase 2 are poor. The decomposition circuitry 26 detects that the vessel segmentations are not well represented by their segmentation in Phase 2. The decomposition circuitry 26 identifies a defect in Phase 2.

With respect to the vessels, Phase 2 is skipped in the animation of the phases in the video data. Therefore, the phase where the vessels aren't well represented is skipped for the vessels, while keeping this frame active for the rest of the heart. The animation of the vessels goes straight from Phase 1 to Phase 3 while the animation of the rest of the heart includes all of Phase 1, Phase 2, Phase 3. The decomposition circuitry 26 outputs a set of video data comprising a sequence of frames in which the representation of the vessels in Phase 2 is omitted, and is replaced by the better representation of the vessels from Phase 1. The rendering circuitry 24 displays an animation that is a rendering of the output video data.

In general, the output video data may omit frames or parts of frames that have been found to include a defect. The defect may comprise a poor segmentation as described above, for example a poor vessel segmentation. In other embodiments, any suitable defect may be identified and the frame or part of frame containing the defect may be omitted. Entire frames may be omitted if they don't have suitable shapes detected.

By using the method of FIG. 9, decomposition into different iso-levels may be used to improve animation even if interpolation is not used. When visualizing a body part which is easily influenced by motion (for example, the coronary artery), dividing the image data into multiple iso levels and/or segmentation may allow the use of only reliable data and the exclusion of poorer data.

Appropriate anatomy (for example, the coronary artery) may be visualized in all time phases. The user may find it more comfortable to view an animation in which the appearance of certain anatomy is consistent. The user may found it easier to view an animation in which frames having a poor representation of anatomy are at least partially omitted.

In embodiments described above, each frame has a single acquisition time. All of the voxels in a given frame may be assumed to have been acquired at the same time.

In some acquisition methods, there may be temporal variation within a single frame. Different parts of a single volume or single image may be captured at different times. An example of a method having temporal variation may be ultrasound. For example, temporal variation within a frame may be present in B mode ultrasound. Temporal variation within a frame may be very prominent in Doppler mode ultrasound, because Doppler mode is typically a slower acquisition mode.

Methods of interpolation described above may be extended to accommodate data in which there is temporal variation within frames.

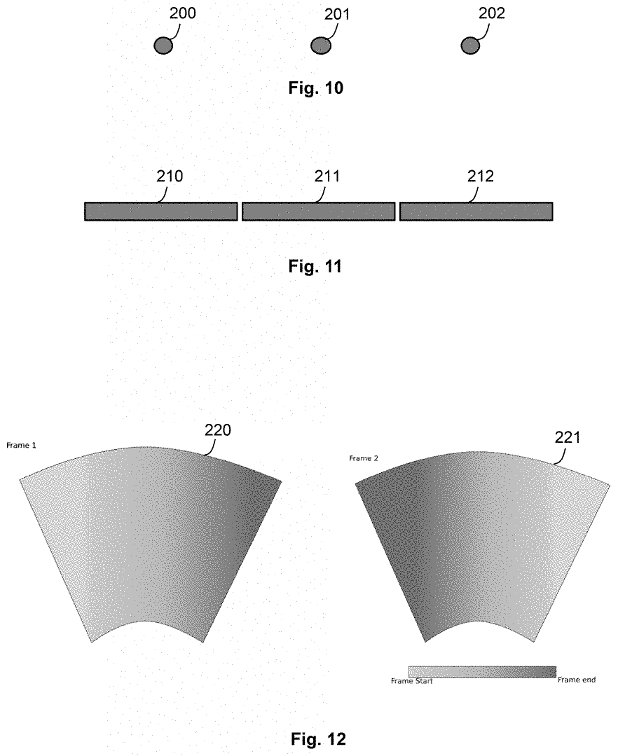

FIG. 10 and FIG. 11 are simple illustrations of different acquisitions. In FIG. 10, three dots 200, 201, 202 represent the acquisition of respective frames. Each acquisition is acquired at a single point in time, which is represented by a dot. The arrangement of dots 200, 201, 202 from left to right is representative of the placement of the acquisitions in time.

Embodiments described above with reference to FIGS. 2 to 9 deal with frames as single points in time, as depicted in FIG. 10. In such embodiments, the interpolation deals with a single time number per volume.

In FIG. 11, three bars 210, 211, 212 represent the acquisition of respective frames. Each acquisition is acquired over an extended time period. Therefore, certain voxels of the first acquisition of bar 210 are acquired before other voxels of the first acquisition of bar 210. The same applies to the other acquisitions of bars 211, 212. Again, time is represented from left to right.

It may be seen from FIG. 11 that some parts of the acquisition of, for example, bar 210 may be closer in time to some parts of bar 211 than to other parts of bar 210. For example, an acquisition at the end of bar 210 is closer to the start of bar 211 than to the start of bar 210.

In some circumstances, pixels or voxels of different frames are not acquired in the same order or direction. FIG. 12 is a schematic illustration of two consecutive ultrasound frames 220, 221. Shading on each of the frames 220, 221 is representative of an order in which different parts of the frames 220, 221 are required. A legend 222 shows that light areas of the frame are obtained towards the start of the acquisition, and darker areas are obtained towards the end of the acquisition. The acquisition of frame 220 starts at the left of the frame and proceeds to the right of the frame. The acquisition of frame 221 starts at the right of the frame and proceeds to the left of the frame.

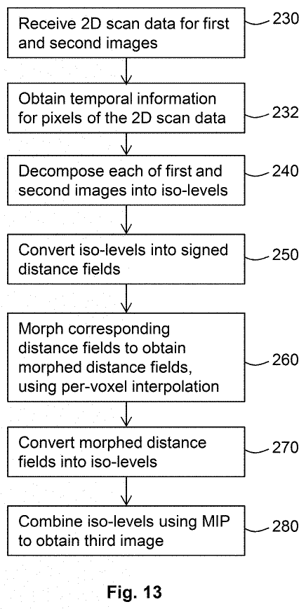

FIG. 13 is a flowchart illustrating an overview a method according to an embodiment in which each frame is acquired over a time period, such that different pixels in a frame may have different acquisition times. In the embodiment of FIG. 13, the frame comprises two-dimensional image data acquired by a two-dimensional scan. In other embodiments, the method of FIG. 13 may be extended to three-dimensional data.

At stage 230 of FIG. 13, the rendering circuitry 24 receives imaging data representative of two frames of a sequence of frames from the memory 20. Each frame has been acquired over a respective extended time period.

At stage 232, the decomposition circuitry 26 extracts temporal information for each pixel in each frame. For example, the decomposition circuitry 26 may extract and store a respective time value for each pixel in a given frame. The time values may be stored in memory 20 or in any appropriate data store.

In order to assign time value to the pixels, a per pixel intraframe timing estimate or measurement is used. The per pixel inter frame timing estimate or measurement may comprise a time of acquisition for a given pixel in a first frame, and a time of acquisition for that pixel in a second frame. The per pixel inter frame timing estimate measurement may comprise a difference in time between the acquisition of the pixel in the first frame and the acquisition of the pixel in the second frame. The difference in acquisition times may be different for different pixels, for example if the pixels are acquired in a different order in the first frame then in the second frame.

In some embodiments, a further data volume or data channel is available which determines an acquisition time point for each pixel. The further data volume or data channel may be stored in the data store 20 or in any appropriate memory. The further data volume or data channel may represent a time at which each of the voxels is acquired. For example, the further data volume or data channel may be obtained from the scanner used to acquire the frames. Temporal information may be reconstructed into an acquisition time volume.

In some acquisition methods, for example in ultrasound Doppler or ultrasound elastography, a sampling process may acquire data values for each voxel in turn. In such acquisitions, individual timings for each voxel may be obtained from knowledge of the timing of each voxel acquisition. An acquisition time volume may comprise a respective timing for each voxel in an acquired volume.

In other acquisition methods, for example in other types of ultrasound acquisition, data may be acquired beam by beam. Each beam may have its own respective acquisition time. In ultrasound, near echoes within a beam arrive earlier than far echoes. Reconstructing an acquisition time volume for such acquisitions may comprise mapping beams onto a volume with a different geometry, which may be a Cartesian geometry.

In some embodiments, an acquisition device model is used that can give an estimate or measurement of the temporal offset of any specific location within a frame. The acquisition device model may be a heuristic model.

In an example, an acquisition method uses an acquisition device comprising a mechanical ultrasound probe. The mechanical ultrasound probe comprises a 2D transducer that scans a set of beams at almost the same time. The 2D transducer pivots up and down using a motor in a back and forth manner. The motor speed is not necessarily constant as the 2D transducer pivots.

An acquisition device model is obtained that provides which angle the 2D transducer is pointed towards at any given time. The acquisition device model also includes any known delays in the emission and reception of individual beams within a 2D slice. If starting conditions are known or can be estimated, the acquisition device model may be used to determine a respective time of acquisition for each voxel in an acquired volume.

At stage 240, the decomposition circuitry 26 receives a set of N pre-defined iso-level values, for example 10 pre-defined iso-level values.

The decomposition circuitry 26 decomposes the first frame into a first plurality of N iso-level images, numbered from 1 to N. Each of the N iso-level images is representative of a respective iso-level surface in the first image. In the present embodiment, each of the iso-level images is obtained by thresholding the first frame using a respective one of the set of N pre-defined iso-level values. In other embodiments, any suitable method may be used to obtain the iso-level images.

The decomposition circuitry 26 decomposes the second frame into a second plurality of iso-level images, using the same method and the same set of N iso-level values that were used for the second plurality of iso-level images.

At stage 250, the decomposition circuitry 26 converts each of the iso-level images into a respective signed distance field. For each iso-level image, the signed distance field is representative of the shape of the iso-level. In other embodiments, any distance transform may be used that is representative of the shape of the iso-level.

In the present embodiment, the method used to decompose each image into a respective plurality of iso-level images and to convert each of the iso-level images into a respective signed distance field is the same as that used in the embodiment of FIG. 2 above. The extraction of signed distance fields is robust to temporal variation. The extraction of the shapes that are used to form the signed distance fields does not need to represent the shape at a fixed time point. It is possible for different parts of the signed distance fields to be associated with different time points.

The output of stage 250 comprises a first plurality of N signed distance fields corresponding to the first plurality of iso-levels obtained from the first frame, and a second plurality of N signed distance fields corresponding to the second plurality of iso-levels obtained from the second frame. The first plurality of signed distance fields and second plurality of signed distance fields are stored temporarily in data store 20 or in any appropriate memory.

In stages 260 to 280 of FIG. 13, the interpolation circuitry 28 uses the signed distance fields to obtain an interpolated image for a time t. In the present embodiment, the time t is a time between a time at which the acquisition of the first frame finished and a time at which the acquisition of the second frame started. In other embodiments, to any suitable time t may be used.

The interpolation circuitry 28 takes into account the different times at which different parts of each image have been acquired.

At stage 260, the interpolation circuitry 28 morphs corresponding signed distance fields to obtain morphed distance fields. For each corresponding iso-level for the first and second frames, a new signed distance field is obtained for time t by interpolating the pixels of the signed distance fields for the first and second frame. A shape blending operation is performed using the temporal location t to calculate the weight of each frame.

In the embodiment of FIG. 13, the shape blending operation takes into account the different times at which different pixels were acquired. In the present embodiment, the interpolation circuitry 28 combines pairs of signed distance fields that correspond to the same iso-level. In other embodiments, signed distance fields for different iso-levels may be morphed together.

For each pixel, the interpolation circuitry 28 retrieves the time value associated with that pixel in the first frame and the time value associated with that pixel in the second frame. The interpolation circuitry 28 interpolates the pixel values to time t using the retrieved time values.

Different interpolation weights are used for pixels acquired at different times. For example, for some pixels the time value for the pixel in the first frame may be closer to time t than the time value for the pixel in the second frame. For other pixels, the time value for the pixel in the first frame may be closer to time t than the time value for the pixel in the second frame. Depending on acquisition order, a difference in the time value in the first frame and the time value in the second frame may be different for different pixels.

The morphing of the signed distance fields may be performed using any suitable interpolation method, for example linear, quadratic or cubic interpolation or any similar smooth interpolation method.

The output of stage 260 is a set of signed distance fields each of which has been morphed such that all pixels are representative of a consistent time value t.

At stage 270, the interpolation circuitry 28 converts each morphed signed distance field back into an iso-level image having an associated iso-level value.

At stage 280, the interpolation circuitry 28 accumulates all of the iso-level images of stage 270 into a single image by selecting maximum intensity as described above in relation to stage 80 of FIG. 2. In other embodiments, any suitable method may be used to combine the iso-level images.

The method of FIG. 13 extends the method of the embodiment described above with relation to FIGS. 2 and 3. By using a per-pixel interpolation, the method of FIG. 2 has the ability to handle and correct temporal variation in the acquired data.

In the embodiment of FIG. 13, the frames comprise two-dimensional image data obtained by two-dimensional scanning. In other embodiments, the frames may comprise three-dimensional image data acquired by three-dimensional scanning or by a plurality of two-dimensional scans. In such embodiments, a per-voxel interpolation may be used instead of a per-pixel interpolation. Temporal upsampling may be used on volumetric data comprising a grid or set of volumetric samples.

Motion correction may be performed in the case of either a volumetric scan using voxels or a 2D scan using pixels, so long as the acquisition time point of each of these elements can be identified to use in the interpolation of a new frame.

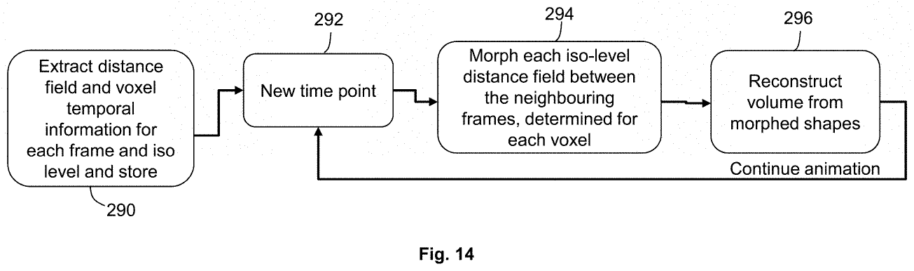

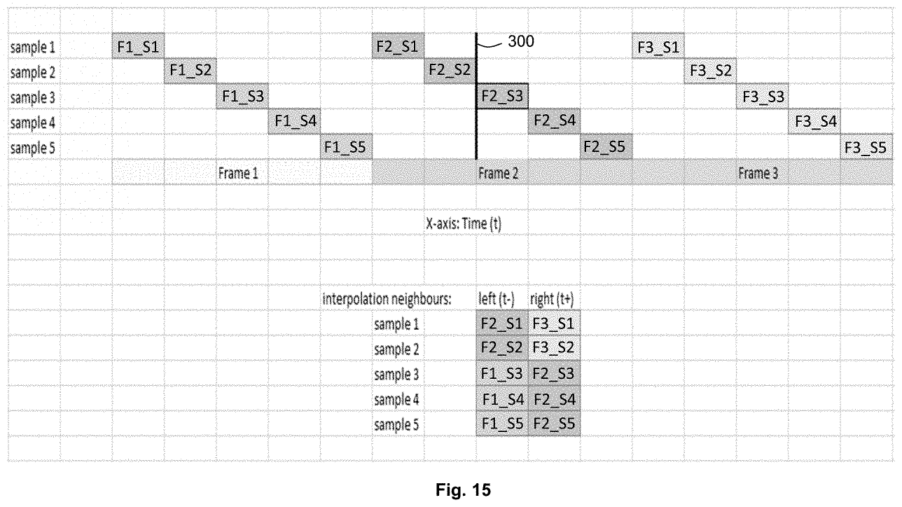

FIG. 14 is a flow chart illustrating in overview a method of providing an animation in accordance with an embodiment in which time variation within frames is taken into account.

At stage 290, the rendering circuitry 24 receives imaging data representative of a sequence of frames from the memory 20. Each frame has been acquired over a respective extended time period. Each frame comprises a respective set of volumetric data.

The decomposition circuitry 26 decomposes each frame into a set of distance fields representing iso levels. A method of decomposition may be similar to methods described above with reference to FIGS. 2, 3 and 13, but applied to volumetric data sets.

The decomposition circuitry 26 decomposes each frame into a respective plurality of N iso-level volumes, and converts each of the iso-level volumes into a respective three-dimensional signed distance field. For each iso-level image, the signed distance field is representative of the shape of the iso-level. In other embodiments, any distance transform may be used that is representative of the shape of the iso-level.

The decomposition circuitry 26 extracts temporal information for each voxel in each frame. In the present embodiment, the decomposition circuitry 26 creates a volume or model of each frame detailing the intra frame timing of the voxels. Any suitable method of obtaining time values for the voxels may be used, for example a measured data channel or estimation using a heuristic model.

At stage 292, the interpolation circuitry 28 moves to a new time point, for example a first time point t. Any suitable time point may be used. The time point may be a time point between frames, or a time point within a time period during which a frame was being acquired.

At stage 294, the interpolation circuitry 28 performs a morphing operation. For each voxel, the interpolation circuitry 28 identifies voxels of two adjacent frames. In one adjacent frame, a value for the voxel was acquired before the first time point t. In the other adjacent frame, the value for the voxel was acquired after the first time point t.

It is noted that in some circumstances, different voxels may have different adjacent frames. When frames are representative of extended time periods instead of individual time points, frame adjacency may be per-voxel rather than consistent for an entire frame.

FIG. 13 is a diagram illustrating a simplified data acquisition scenario. A sequence of three consecutive frames are labelled F1, F2, F3 in the order of their acquisition. In the simplified scenario, each of the frames comprises five samples acquired over a time period, such that each sample has a different acquisition time. Frame F1 comprises samples labelled F1_S1 . . . F1_S5 in order of acquisition time. Frame F2 comprises samples labelled F2_S1 . . . F2_S5. Frame F3 comprises samples labelled F3_S1 . . . F3_S5. Each of the frames is acquired in the same order. Labelling is such that all samples labelled with S1 correspond to the same sample point S1 (for example, the same pixel or voxel), all samples labelled with S2 correspond to the same sampling point S2, and so on.

A first time point 300 falls between a time of acquisition of sample F2_S2 and a time of acquisition of sample F2_S3 of frame F2.

To obtain an interpolated frame 11 for the first time point, the interpolation circuitry 28 determines for each sample point in the interpolated frame 11 a left interpolation neighbor that is the immediately preceding sample for that sample point, and a right interpolation neighbor that is the immediately following sample for that sample point.

For sample point S1, the immediately preceding sample is F2_S1 and the immediately following sample is F3_S1. For sample point S2, the immediately preceding sample is F2_S2 and the immediately following sample is F3_S2. For S1 and S2, the frames adjacent to first time point 300 are F1 and F2.

For sample point S3, the immediately preceding sample is F1_S3 and the immediately following sample is F2_S3. For sample point S4, the immediately preceding sample is F1_S4 and the immediately following sample is F2_S4. For sample point S5, the immediately preceding sample is F1_S5 and the immediately following sample is F2_S5. For S1, S2 and S3 the frames adjacent to first time point 300 are F1 and F2.

In the case of a Doppler acquisition, a sampling pattern of point samples may be selectable by an acquisition algorithm. Usually a grid of points is used, but sometimes randomized samples may be used. In the case of randomized samples, it may be the case that some points are only included in a subset of frames and not in all frames. Again, different voxels may have different adjacent frames.

The interpolation circuitry 28 morphs each paired distance function by interpolating values for adjacent frames on a per-voxel basis. The interpolation for each voxel is weighted using the time values for that voxel in the identified adjacent frames.

The interpolation circuitry 28 converts each morphed signed distance field back into an iso-level image having an associated iso-level value.

An output of stage 294 is a set of iso-level volumes. In each of the iso-level volumes, all of the voxel values are representative of the same first time point t. By performing the morphing operation on a per-voxel basis, there is an opportunity to correct temporal variance within each frame.

At stage 296, the interpolation circuitry 28 reconstructs an intermediate volume from the morphed signed distance fields. In the present embodiment, the interpolation circuitry 28 accumulates the iso-level volumes into a single volume by selecting the maximum intensity between a current and a previous morphed iso-level. In other embodiments, any suitable method may be used to combine the iso-level volumes.

The flow chart returns to stage 292 and a new time point is selected. The animation continues at the new time point.

Interpolation may be performed at any suitable times and with any suitable interval between interpolated frames. The intra frame temporal variation does not need to be the same in different frames. Voxels are treated individually at each time point.

The rendering circuitry 24 renders a respective image from each of the reconstructed volumes and displays an animation of the rendered images. The rendering circuitry 24 displays an animation using the image data. In the animation, images are motion corrected. At each time point, intra frame temporal variation may be reduced or eliminated by performing a morphing process that is specific to that time point and takes into account time differences between voxels. The time points may be chosen such that the animation appears smooth to the human eye, for example by choosing a frame rate that is greater than 20 fps.

In some embodiments, the decomposing of all frames is performed in advance and cached. Cached data may be compressed by removing data values for voxels that, given the surrounding frames, will not affect the interpolated end-result volume. In other embodiments, the decomposing may be performed on a just in time basis.

The methods of FIGS. 13 and 14 may provide an improved method of motion correction. The method may be less resource intensive than some methods of motion correction. The method may be built in to an interpolation method that may also be desirable for other reasons, for example to present a smoother animation to a user.

In the embodiments above, the steps of rendering, extracting iso-levels, extracting signed distance fields, morphing signed distance fields, converting the morphed signed distance fields into iso-levels and combining the iso-levels are all described as separate steps. In other embodiments, the functions of two or more of these steps may be combined into a single step. In further embodiments, one or more of the steps described above may be split into multiple steps.

In embodiments above, various data sets (for example, data sets that are representative of images, iso-level images and/or signed distance fields) are stored in data store 20. In other embodiments, some or all the data sets may be stored in any suitable data store. Some or all of the data sets may be cached locally. Some or all of the data sets may be used directly without being stored.

Methods are described above with relation to medical imaging, in particular medical imaging of the heart. In other embodiments, methods described above may be used in relation to medical imaging of any anatomical region of any human or animal subject. References to medical may include veterinary. In further embodiments, methods described above may be applied to any suitable type of imaging, which may not be medical. For example, methods described above may be used to visualize results of fluid simulation in the automobile sector or aerospace sector. Methods described above may be used in volume rendering for oil and gas. Methods described above may be used for volume rendering of sonar.

Certain embodiments provide a medical imaging method comprising a set (minimum 2) of volumes representing a temporal location, a set of pre-defined iso-levels, and a temporal location t in between the volume frames, in which an interpolated volume is created by: (1) decomposing the volume into a set of iso-levels and generating a signed distance field representing the shape of the iso-level; (2) for each corresponding iso-level for the frames in the interpolation neighborhood, a new signed distance field is created by interpolating the distance field, representing a shape blending operation using the temporal location t to calculate the weight of each frame; (3) the resulting set of morphed distance fields are then accumulated back into a destination volume by selecting the maximum intensity of each morphed iso-level, for each voxel.

Step 1 may be done ahead of time and the result cached. Step 1 may be done just in time. The result subset required for the interpolation t may be cached. The interpolation method used for the signed distance field blend operation may be either linear, quadratic, cubic or similar smooth interpolation method. The iso-levels between the frames may be different to account for inconsistencies in the intensity of the captured tissue. Segmentation and/or clipping may be allowed to constrain each iso-level. Iso-levels from multiple volumes may be included and reconstructed into a motion interpolated fusion volume. Signed distance field, static or animated, may be included to incorporate external shapes. Iso-levels may be selected manually. Iso-levels may be selected by selecting the most visible parts of the volume calculated using a visibility histogram based on the view presented to the user. A deep neural network may be used to classify the iso-levels causing the least amount of differences to the final image at key frames.

The system may be applied in near-real time by: waiting for the volumes to come in from an acquisition process and when they do to decompose and store the set of signed distance fields associated with each iso-level; provide a set of interpolated in between the last set of volume frames using the method and at a speed based on the expectation of when the next frame will appear. This may give the appearance of smooth real time motion offset by acquisition time step behind the true real time results.

Certain embodiments provide an image processing apparatus comprising processing circuitry configured to: acquire first image data corresponding to first timing and second image data corresponding to second timing which is different timing of the first timing, transform the first image data and the second image data into a plurality of first transformed data and a plurality of second transformed data based on a parameter of image data, transform the first transformed data and the second transformed data into first signed distance field and second signed distance field, select a combination of the first signed distance field and the second signed distance field based on the parameter, generate morphed data based on the combination of the first signed distance field and the second signed distance field, generate up-sampled image data between the first image data and the second image data, by processing the plurality of the morphed data generated in accordance with the parameter values.

Certain embodiments provide an image processing apparatus comprising processing circuitry configured to: acquire first image data corresponding to first timing and second image data corresponding to second timing which is different timing of the first timing, transform the first image data and the second image data into a plurality of first transformed data and a plurality of second transformed data based on a parameter of image data, specify a defect of image data based on the first transformed data and the second transformed data, generate video data based on the specify procedure.

Certain embodiments provide a medical imaging method comprising a set (minimum 2) of volumes, a set of pre-defined iso-levels and a temporal location t in between the volume frames, in which an interpolated volume is created by
1. Decomposing the volume into a set of ISO-levels and generating a signed distance field representing the shape of the iso-level;
2. For each corresponding iso-level for the frames in the interpolation neighbourhood, a new signed distance field is created by interpolating the distance field, representing a shape blending operation using the temporal location t by also considering the intra frame acquisition time for the voxel neighbourhood; and
3. The resulting set of morphed distance fields are then accumulated back into a destination volume by selecting the maximum intensity of each morphed iso-level, for each voxel.

The temporal information may be reconstructed into a voxel acquisition time volume. The temporal information may be a heuristic model predicting the voxel acquisition time based in knowledge about the acquisition device.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. An image processing apparatus comprising processing circuitry configured to:
acquire first image data that is representative of a subject at a first time and second image data that is representative of the subject at a second, different time;
process the first image data based on a parameter of the image data to obtain a plurality of transformed first data sets, each of the transformed first data sets corresponding to a respective value for the parameter;
process the second image data based on the parameter of the image data to obtain a plurality of transformed second data sets, each of the transformed second data sets corresponding to a respective value for the parameter;
transform each of the transformed first data sets to obtain a respective first distance transform;
transform each of the transformed second data sets to obtain a respective second distance transform;
select a combination of at least one of the first distance transforms and at least one of the second distance transforms based on the parameter;
generate at least one morphed distance transform based on the combination; and
process the at least one morphed distance transform to obtain upsampled image data that is representative of the subject at a third time, wherein the third time is between the first time and the second time.

2. An apparatus according to claim 1, wherein each first distance transform comprises a respective first signed distance field, each second distance transform comprises a respective second signed distance field, and the at least one morphed distance transform comprises at least one morphed distance field.

3. An apparatus according to claim 1, wherein the parameter of the image data comprises intensity.

4. An apparatus according to claim 3, wherein processing the first image data set to obtain the plurality of transformed first data sets comprises thresholding the first image data using different values of intensity, and processing the second image data set to obtain the plurality of transformed second data sets comprises thresholding the second image data using different values of intensity.

5. An apparatus according to claim 1, wherein each first distance transform is representative of a shape of a respective iso-level of the first data set, and each second distance transform is representative of a shape of a respective iso-level of the second data set.

6. An apparatus according to claim 1, wherein the combination of at least one of the first distance transforms and at least one of the second distance transforms comprises pairs of first distance transforms and second distance transforms, each pair having a common value for the parameter, and wherein generating at least one morphed distance transform based on the combination comprises interpolating between each of the pairs.

7. An apparatus according to claim 1, wherein the combination of at least one of the first distance transforms and at least one of the second distance transforms comprises pairs of first distance transforms and second distance transforms, wherein at least some of the pairs have a different value for the parameter in respect of the first distance transform than in respect of the second distance transform, and wherein generating at least one morphed distance transform based on the combination comprises interpolating between each of the pairs.

8. An apparatus according to claim 1, wherein the combination of the at least some of the first distance transforms and at least some of the second distance transforms is weighted in dependence on the difference in time between the third time and first time, and the difference in time between the third time and second time.

9. An apparatus according to claim 1, wherein processing the morphed distance transforms to obtain upsampled image data comprises selecting a maximum intensity of each morphed distance transform for each voxel.

10. An apparatus according to claim 1, wherein the processing of the first image data and second image data based on a parameter of the image data is performed in advance and cached.

11. An apparatus according to claim 1, wherein the first image data and second image data each comprise data from a respective plurality of image acquisitions, and wherein the upsampled image data comprises fusion image data.

12. An apparatus according to claim 1, further comprising incorporating into the first distance transforms and second distance transforms a representation of an object that is not part of the subject.

13. An apparatus according to claim 1, wherein the processing of the first image data and second image data based on the parameter comprises selecting a plurality of values for the parameter.

14. An apparatus according to claim 13, wherein the selecting of the plurality of values for the parameter is performed by a trained model.

15. An image processing method comprising:
acquiring first image data that is representative of a subject at a first time and second image data that is representative of the subject at a second, different time;
processing the first image data based on a parameter of the image data to obtain a plurality of transformed first data sets, each of the transformed first data sets corresponding to a respective value for the parameter;
processing the second image data based on the parameter of the image data to obtain a plurality of transformed second data sets, each of the transformed second data sets corresponding to a respective value for the parameter;
transforming each of the transformed first data sets to obtain a respective first distance transform;
transforming each of the transformed second data sets to obtain a respective distance transform;
selecting a combination of at least one of the first distance transforms and at least one of the second distance transforms based on the parameter;
generating at least one morphed distance transform based on the combination; and
processing the at least one morphed distance transform to obtain upsampled image data that is representative of the subject at a third time, wherein the third time is between the first time and the second time.

16. An image processing apparatus comprising processing circuitry configured to:
acquire first image data that is representative of a subject at a first time and second image data that is representative of the subject at a second, different time;
process the first image data based on a parameter of the image data to obtain a plurality of transformed first data sets, each of the transformed first data sets corresponding to a respective value for the parameter;
process the second image data based on the parameter of the image data to obtain a plurality of transformed second data sets, each of the transformed second data sets corresponding to a respective value for the parameter;
identify a defect of the first image data and/or the second image data, wherein the identifying of the defect is based on the first transformed data sets and the second transformed data sets; and
generate video data based on the identifying of the defect.

17. An apparatus according to claim 16, wherein the video data comprises a plurality of frames each obtained from respective image data, and the generating of the video data comprises omitting from the video data at least part of a video frame based on the image data in which the defect is identified.

18. An apparatus according to claim 16, wherein the identified defect is a defect in a segmentation of at least one object represented in the first image data and/or second image data.

19. An apparatus according to claim 16, wherein the generating of the video data comprises obtaining at least one upsampled frame of the video data using an upsampling procedure, and wherein at least part of the image data in which the defect is identified is omitted from the upsampling procedure.

20. An image processing method comprising:
acquiring first image data that is representative of a subject at a first time and second image data that is representative of the subject at a second, different time;
processing the first image data based on a parameter of the image data to obtain a plurality of transformed first data sets, each of the transformed first data sets corresponding to a respective value for the parameter;
processing the second image data based on the parameter of the image data to obtain a plurality of transformed second data sets, each of the transformed second data sets corresponding to a respective value for the parameter;
identifying a defect of the first image data and/or the second image data, wherein the identifying of the defect is based on the first transformed data sets and the second transformed data sets; and
generating video data based on the identifying of the defect.

21. An image processing apparatus comprising processing circuitry configured to:
obtain first image data that is representative of a subject, wherein the first image data comprises image data acquired over a first time period, and second image data that is representative of the subject, wherein the second image data comprises image data acquired over a second, different time period;
obtain temporal information indicative of a respective time of acquisition for each pixel or voxel of the first image data and second image data;
process the first image data based on a parameter of the image data to obtain a plurality of transformed first data sets, each of the transformed first data sets corresponding to a respective value for the parameter;
process the second image data based on the parameter of the image data to obtain a plurality of transformed second data sets, each of the transformed second data sets corresponding to a respective value for the parameter;
transform each of the transformed first data sets to obtain a respective first distance transform;
transform each of the transformed second data sets to obtain a respective second distance transform;
select a combination of at least one of the first distance transforms and at least one of the second distance transforms based on the parameter;
generate at least one morphed distance transform based on the combination, wherein the generating of the at least one morphed distance transform is in dependence on the temporal information; and
process the at least one morphed distance transform to obtain upsampled image data that is representative of the subject at a target time.

22. An apparatus according to claim 21, wherein the combination of at least one of the first distance transforms and at least one of the second distance transforms comprises pairs of first distance transforms and second distance transforms, and wherein generating at least one morphed distance transform based on the combination comprises interpolating between each of the pairs based on the temporal information.

23. An apparatus according to claim 22, wherein interpolating between each of the pairs based on the temporal information comprises determining per-pixel or per-voxel weights based on the temporal information.

24. An apparatus according to claim 21, wherein the combination of the at least some of the first distance transforms and at least some of the second distance transforms is weighted on a per-pixel or per-voxel basis in dependence on a difference in time between the target time and a respective acquisition time for each pixel or voxel in the first image data, and a difference in time between the target time and a respective acquisition time for each pixel or voxel in the second image data.

25. An apparatus according to claim 21, wherein the processing circuitry is further configured to:
acquire third image data that is representative of the subject and is acquired over a third, different time period;
process the third image data based on a parameter of the image data to obtain a plurality of transformed third data sets, each of the transformed third data sets corresponding to a respective value for the parameter; and
transform each of the transformed third data sets to obtain a respective third distance transform;
wherein the combination of the at least one of the first distance transforms and the at least one of the second distance transforms further comprises at least one of the third distance transforms.

26. An apparatus according to claim 25, wherein the generating of the at least one morphed distance transform is performed on a per-pixel or per-voxel basis, and comprises combining data values from the at least one of the first distance transforms and at least one of the second distance transforms for some pixel or voxel positions, and combining data values from the at least one of the first distance transforms and at least one of the third distance transforms for other pixel or voxel positions.

27. An apparatus according to claim 21, wherein the time information comprises an acquisition time volume.

28. An apparatus according to claim 21, wherein the obtaining of the time information comprises estimating pixel or voxel acquisition times using knowledge of an acquisition device used to acquire the first image data and second image data.

29. An image processing method comprising:
obtaining first image data that is representative of a subject, wherein the first image data comprises image data acquired over a first time period, and second image data that is representative of the subject, wherein the second image data comprises image data acquired over a second, different time period;
obtaining temporal information indicative of a respective time of acquisition for each pixel or voxel of the first image data and second image data;
processing the first image data based on a parameter of the image data to obtain a plurality of transformed first data sets, each of the transformed first data sets corresponding to a respective value for the parameter;
processing the second image data based on the parameter of the image data to obtain a plurality of transformed second data sets, each of the transformed second data sets corresponding to a respective value for the parameter;
transforming each of the transformed first data sets to obtain a respective first distance transform;
transforming each of the transformed second data sets to obtain a respective second distance transform;
selecting a combination of at least one of the first distance transforms and at least one of the second distance transforms based on the parameter;
generating at least one morphed distance transform based on the combination, wherein the generating of the at least one morphed distance transform is in dependence on the temporal information; and
processing the at least one morphed distance transform to obtain upsampled image data that is representative of the subject at a target time.
